(19) United States
(12) Patent Application Publication
(10) Pub. No.: US 2002/0122826 A1
(43) Pub. Date: Sep. 5, 2002

(54) Solid peptide preparations for inhalation and their preparation

(76) Inventors: Rosario Lizio, Buttelborn (DE); Michael Damm, Rodermark (DE); Werner Sarlikiotis, Peania (GR); Elisabeth Wolf-Heuss, Mosbach (DE)

Correspondence Address: Goodwin Procter L.L.P., 599 Lexington Avenue, 40th floor, New York, NY, 10022, US

(21) Appl. No.: 09/944,060

(22) Filed: Aug. 31, 2001

(30) Foreign Application Priority Data
Sep. 1, 2000 (DE) 100 43 509.2

(51) Int. Cl. A61K 9/14; B02C 11/08

(52) U.S. Cl. 424/489; 241/18

(57) ABSTRACT

The invention relates to solid pharmaceutical preparations, in particular for inhalatory administration in mammals, their preparation and their use such as, for example, in powder inhalers.

The invention relates to solid pharmaceutical preparations, in particular for inhalatory administration in mammals, their preparation and their use such as, for example, in powder inhalers.

The invention relates to the preparation of pharmaceutical formulations and to their preparation processes in which micronized powder or powder mixtures consisting of active compounds or active compound/excipient mixtures or excipients or excipient mixtures are applied without the use of binders to carrier materials or carrier material mixtures made of various excipients. In addition, the invention relates to a process for the preparation of the suspensions needed for these pharmaceutical formulations or the micronized powders of active compounds or excipients or active compound/excipient mixtures isolated therefrom.

Inhalatory therapy is normally carried out by inhalation of aerosols. Drops or solid particles can be suspended in air and inhaled. Aerosols of solid particles can be obtained from a suspension in propellant (MDI) or from a powder. For macromolecular substances, micronization and application to carrier materials (such as, for example, lactose, maltose, trehalose) presents the greatest difficulty (A. K. Banga Therapeutic Peptides and Proteins: Formulation, Processing, and Delivery Systems. Lancaster, Basle: Technomic Publishing Co., Inc.; 1996). The customary micronization processes such as spray drying, the use of an air-jet mill or a ball mill are less suitable for such substances, in particular because of stability and contamination problems (Y. -F. Maa, P. -A. Nguyen, T. Sweeney, S. J. Shire and C. C. Hsu. Protein inhalation powders: spray drying vs freeze drying. Pharm. Sci., 16 (2): 249-254 (1999); Y. -F. Maa, P. -A. Nguyen, J. D. Andya, N. Dasovich, T. Sweeney, S. J. Shire and C. C. Hsu. Effect of spray drying and subsequent processing conditions on residual moisture content and physical/biochemical stability of protein inhalation powder. Pharm. Res., 15(5) 768-775 (1998)). The micronization of active compounds for inhalation purposes is necessary in order to produce particles which are in the “respirable” (inhalable) range (<10 μm) (The United States Pharmacopeia. Twenty-third revision. US Pharmacopeial Convention Inc., Rockville, Md., 1995). This applies in particular when a systemic action is to be achieved by inhalatory administration. In this case, the particles must be “alveolar-respirable” (preferably between 0.5 and 3 μm) (A. K. Banga Therapeutic Peptides and Proteins: Formulation, Processing and Delivery Systems. Lancaster, Basle: Technomic Publishing Co., Inc.; 1996; A. MacKellear & N. Osborne. Breathing new life into drug delivery. Manufact. Chemist. 8: 31-33 (1998)). The micronization of active compounds by means of ball mills or bead mills are already long-known processes [sic]. The disadvantage of these processes are [sic] normally the high temperature development and the severe abrasion in the system which leads to stability problems and product contamination. The contamination problems, however, remain unchanged even at low temperatures as long as conventional materials, for example glass, tungsten or stainless-steel balls or beads are used for the parts contacting the product. The temperature development in the grinding process is serious, in particular for sensitive substances such as peptides and proteins, since it can lead to the loss of biological action.

Bead mills have in fact already been used in the pharmaceutical field for the preparation of suspensions in liquid propellant (chlorofluorohydrocarbon) for metered-dose aerosols, but without indication of product impurities (A. L. Adjei, J. W. Kesterson and E. S. Johnson. European patent application. LARH Analog formulation. Public. No. 0510731A1, 1987; A. L. Adjei, E. S. Johnson and J. W. Kesterson. United States Patent LHRH Analog formulation. U.S. Pat. No. 4,897,256; Date: Jan. 30, 1990). A bead mill has likewise been used for the preparation of nanosuspensions in order to achieve an improvement in the solubilities of poorly soluble substances (R. H. Muller, R. Becker, B. Kruss, K. Peters. United States Patent. Pharmaceutical nanosuspensions for medicament administration as system with increased saturation solubility and rate of solution. U.S. Pat. No. 5,858,410; Date Jan. 12, 1999). However, up to now use of the bead mill at low temperature in, for example, liquid hydrofluoroalkanes such as, for example, TG134a or TG227, or other liquids has not been described in order to prepare pure, dry, micronized active compounds.

For the preparation of powder formulations for inhalation purposes, a further additional phase is still also needed: the micronized powder has to be mixed here with a carrier material, for example lactose, dextrose, maltose, trehalose, as described in the patent specification WO96/02231, ASTA-Medica AG and in the article P. Lucas, K. Anderson, J. N. Staniforth, Protein deposition from drypowder inhalers: Fine particle multiplets as performance modifiers. Pharm. Res. 15(4) 562-569 (1998), in order to obtain a flowable powder, a precise meterability of the formulation from a powder inhaler and a good dispersion of the active compound. This process is normally carried out with the aid of mixers such as described in WO96/02231, by means of tumble mixers (for example Turbula) [sic] after prior compulsory sieving and sieving through, for example, stainless steel sieves in order to achieve a distribution of the components in the total mass which is as uniform as possible. It may also be that, for example, in the case of very small active compound particles, for example particles of 0.1-5 μm, long mixing times are necessary for, for example, a cetrorelix/lactose mixture in order to obtain a readily dispersible formulation. A ready-to-use powder formulation is thus only obtainable after a number of sieving and mixing actions. This applies particularly when, combination preparations containing, for example, various active compounds or active compound mixtures or active compound/excipient mixtures such as, for example, formoterol with budesonide are to be prepared in the ratio, for example, 1 part of formoterol to 4 to 70 parts of budesonide, in particular 30 to 36 parts of budesonide, particularly preferably in the ratio 1 to 32.5 (see WO98/15280 and WO93/11773).

or when the loading of the carrier material with the active compound or the active compound mixtures or active compound/excipient mixtures is very low, preferably <4.5%, more preferably <2%, but most preferably <0.5%.

In addition, it is possible to employ for the preparation of micronized powders for inhalatory or other purposes or powder formulations for inhalation, for example: M3 antagonists such as, for example, LAS34273 (also known under the name LAS W 330, anticholinergic, from Almirall), tiotropium (anticholinergic, from Boehringer Ingelheim), ipratropium, oxitropium, flutropium, glycopyrrolates (anticholinergic), APC-366 (mast cell trytase inhibitor, Arris), loteprednol (steroid), AWD-12-281 (PDE-IV), viozan (dual beta-2 and dopamine D2 agonist COPD, Astra Zeneca), IPL 576,092 (Aventis), RPR 106-541 (steroid, Aventis), RP73401 (PDE-IV, Aventis), IL-4r (IL-4 receptor, Immunex/Aventis), BAY 16 9996 (IL-4 receptor antagonist, Bayer), ciclesonide (steroid, Byk-Gulden), romiflulast (PDE-IV inhibitor, Byk-Gulden), D-4418 (PDE-4, Darwin), EpiGenRx (adenosine A1, antisense, EpiGenesis), FR173657 (bradykinin antagonist, Fujisawa), FK888 (NK1 antagonist, Fujisawa), Olizumab E25 (or rhuMAB-E25, Norvatis [sic]/Genentech), tobramycin (CF, PathoGenesis), peptide vaccine (peptide vaccine, Peptide Therapeutics), andolast (mast cell stabilizer, Rotta Research), foropafant (PAF antagonist, Sanofi), Saredudant (NK2 antagonist, Sanofi), SCH 55700 (antibody II-5, Shering [sic] Plough), R,R-formoterol, Sepracor), T-440 (PDE IV, Tanabe), PACAP 1-27 (adenylate cyclase activ., University of California).

According to one aspect of the invention, the object thus consisted in obtaining micronized powders (i.e. fine-particulate powders having particle sizes in the nano- to micrometer range), in particular of active compounds. A further object consisted in simplifying the application of one or more fine-particulate powders to one or more carrier materials or generally firstly making it possible to achieve a more uniform distribution of micronized powders on the carrier material or the carrier materials, to achieve a better dispersibility and, for example, to reduce the contamination risks with respect to product and personal protection and also to achieve a shortening of the preparation time.

A particular problem lies, in particular, in the preparation of powder preparations in which a solid active compound combination is to be applied to a carrier material.

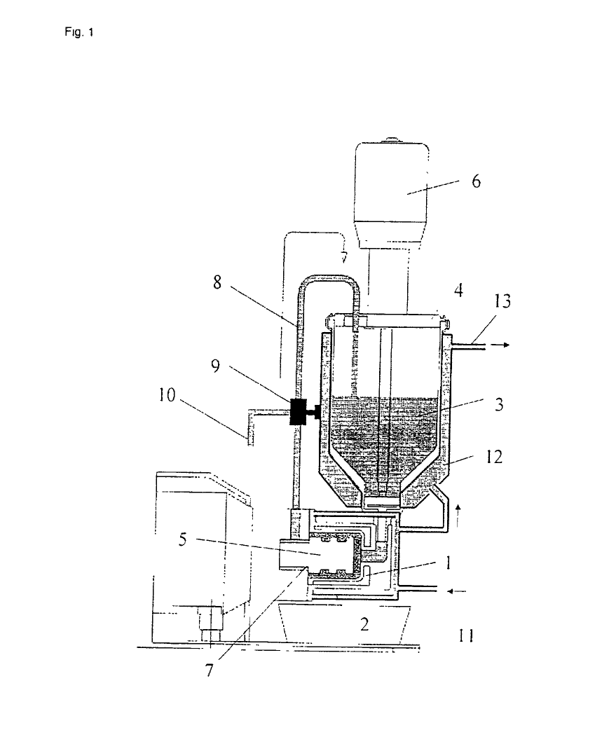

The object of preparing a solid powder formulation has now been achieved by first micronizing a sensitive model substance, for example cetrorelix acetate, as a suspension in liquid propellant, for example in TG227 at temperatures down to <−60° C. to a particle size distribution of 0.1-0.5 μm d(10%) to 5-10 μm d(90%), preferably 0.1-5 μm, preferably to 0.2 d(10%) −4 μm d(90%), particularly preferably to 0.3-0.5 d(10%) −3 μm d(90%) in a bead mill modified for low temperatures (FIG. 1 and example 1) and mixing the suspensions thus obtained with various, for example, lactoses, trehaloses, dextroses having various particle sizes from 10 to 500 μm, preferably 10 to 700 μm, more preferably 10 to 900 μm, and finally obtaining the dry powder formulations for inhalation purposes (e.g. for DPI or MDPI) by evaporation of the propellant or suspending medium by means of a rotary evaporator in the course of <3 h, preferably <2 h, more preferably <1.5 h.

For local or topical administration, particle sizes of between approximately 0.5-10 μm are preferred.

The bead mill necessary for the procedure according to the invention was manufactured by VMA-Getzmann and modified according to our requirements. The basic model (for operation in the positive temperature range) is already commercially obtainable (FIG. 1).

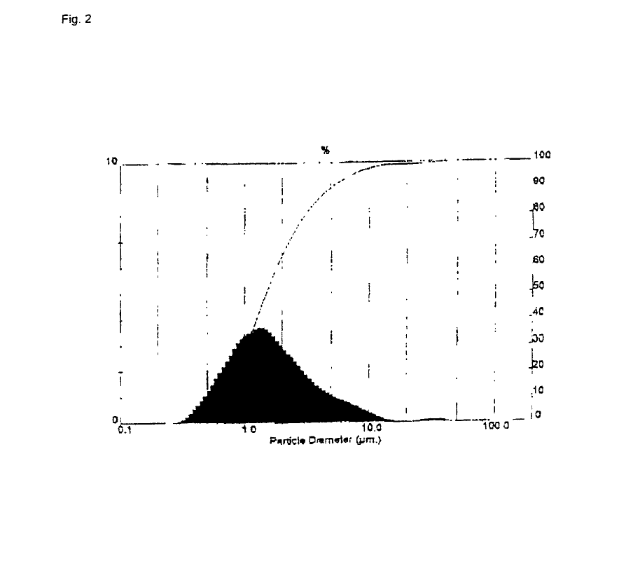

The application areas of this mill are normally the preparation of dye dispersions and ceramic pastes for dental applications. Until now, no micronized powders for pharmaceutical applications are known using this process. The apparatus consists of a grinding chamber (FIG. 1-1) in which, for example, silicon nitride beads, iridium- or yttrium-stabilized ZrO₂ beads (FIGS. 1-2) having bead diameters of, for example, 0.2 to 2 mm and the particles to be comminuted, for example in the form of a suspension (FIGS. 1-3) or as a solid, are introduced via a stainless-steel reservoir fixed to the grinding chamber (FIGS. 1-4). The grinding beads are moved in a circle in the grinding chamber consisting of zirconium dioxide ceramic by means of a “bead mill insert” consisting of zirconium dioxide ceramic (FIGS. 1-5). By means of this, the particles in the suspension are comminuted between the “beads”. The speed of rotation is preferably between 1 m/sec to [sic] 14 m/sec. The suspension is pumped back through the grinding chamber via the return (FIGS. 1-8) into the storage container (FIGS. 1-4) by means of, for example, a centrifugal pump (FIGS. 1-6) and thus kept in circulation. The grinding efficiency and grinding time in order to achieve the desired particle size distribution is dependent on the grinding chamber size, the rate of rotation of the grinding rotor (bead mill insert), the size and amount of grinding beads, the product viscosity or the viscosity of the suspensions, and the particle hardness. The following applies: the more viscous, the better the grinding. The following customarily additionally applies: the harder or more brittle, the better the grinding. The fine suspension obtained is then separated off from the grinding beads through a slot sieve (FIGS. 1-7) having a slot width of, for example, 0.1 to 0.5 mm. By means of the three-way tap (FIGS. 1-9) situated on the return (FIGS. 1-8), the ready-to-use suspension can be pumped via the outlet tube (FIGS. 1-10) for further processing, for example, into a mixer reactor (e.g.: Broglie) in order there to obtain the powder or a ready-to-use powder formulation with, for example, lactose, for example by evaporating the suspending medium. For cooling of the suspensions, for example, 96% ethanol is pumped into the cooling jacket (FIGS. 1-12) of the bead mill via the cooling agent feed (FIGS. 1-11). The outlet (FIGS. 1-13) and the cooling agent feed (FIGS. 1-11) is [sic], for example, connected to a recirculating condenser.

On the one hand, the suspensions obtained were evaporated in an evaporator flask and slow rotation by means of, for example, a rotary evaporator. The powders were either only allowed to stand at RT in order to allow propellant or residues of suspending agent to outgas or a vacuum was applied for a few minutes in order to obtain a pure dry powder or mixtures. On the other hand, the ready-to-use suspensions were added directly to carrier materials or mixtures and the liquid propellant or the suspending medium or mixtures was/were then evaporated to dryness with rotation in the flask and propellant or residues of suspending agent were removed from the mixtures by outgassing at suitable temperatures or by applying vacuum.

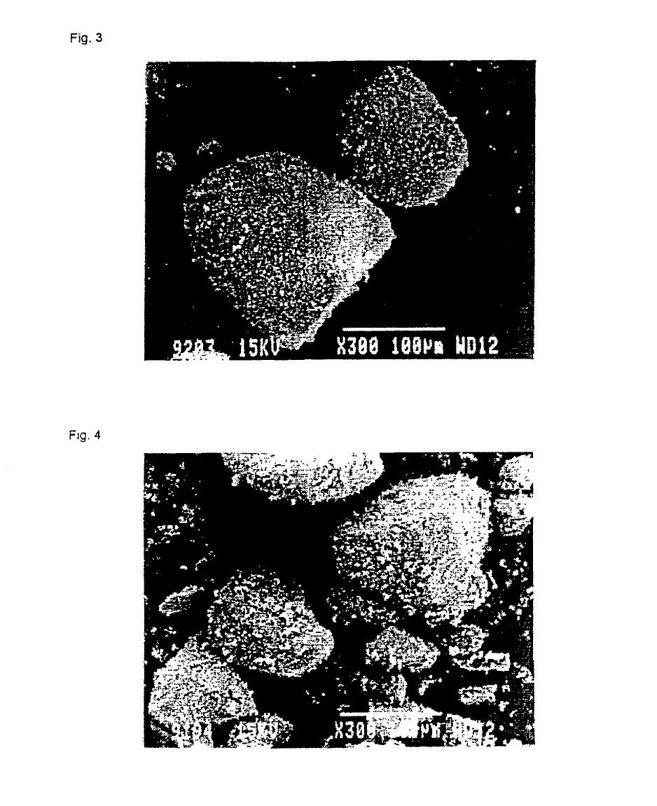

It seems a likely supposition here that the particles or powder mixtures thus prepared agglutinate with one another. And thus none or only a small amount of a powder or of a powder mixture necessary for, for example, inhalatory purposes could be obtained. However, it was surprisingly found that the powder formulations thus prepared showed comparable or better dispersions of active compound and aerodynamic properties at a shorter or the same preparation time of <1.5 h relative to the conventional dry mixing process and powder formulations which was [sic] only prepared by dry mixing processes (see also REM absorption from example 2, FIGS. 3 and 4) and particle size distribution from example 1, FIG. 2 and also the respirable fractions determined as shown in tab. 1).

TABLE 1

| | Respirable fraction, determined by | |
|---|---|---|
| | means of cascade impactor (multi-stage | |
| | liquid impinger, according to Pharm | |
| | Eur.) from ASTRA (explanation: cut-off | |
| | diameter of stage 2 = 12.04 μm, | |
| | stage 3 = 6.30 μm, | |
| | stage 4 = 2.87 μm and | |
| | stage 5 = 1.57 μm) | |
| | Cascade 3-5, | |
| | in % (˜local and | Cascade 4-5, in % |
| Formulation | systemic action) | (˜systemic action) |
| Example 2, cetrorelix, | 41% (n = 1) | 32.2% (n = 1) |
| suspension (SpheroLac 100) | | |
| Comparison example 2a | 40% (n = 2) | n.a. |
| dry (SpheroLac 100) | | |
| Example 3, cetrorelix | 48% (n = 2) | n.a. |
| suspension + turbula (CapsuLac | | |
| 60) | | |
| Comparison example 3a | 32% (n = 2) | n.a. |
| cetrorelix, dry (CapsuLac 60) | | |
| Example 4 | 46% (n = 6) | 37% (n = 6) |
| cetrorelix, suspension (CapsuLac | | |
| 60) | | |
| Comparative example 3a | 32% (n = 2) | n.a. |
| Example 5 | 35% (n = 6) | 23% (n = 6) |
| budesonide, suspension | | |
| (CapsuLac 60) | | |
| Comparative example 5a | 33% (n = 6) | n.a. |
| budesonide, dry (CapsuLac 60) | | |

n.a. = not analyzed
Explanation for table 1: suspension here means: prepared by means of application process, e.g. via suspending in TG227 and subsequent evaporation. Dry here means: preparation by the conventional dry-mixing process. Turbula here means: subsequent mixing of the dry mixture obtained from the application process.

By the process according to the invention, it is possible to micronize solid substances such as, for example, all pharmaceutically active substances, excipients, excipient mixtures and active compound/excipient mixtures, temperature- and oxidation-sensitive substances such as, for example, physiologically active peptides and proteins, in particular LHRH analogs, with or without additional liquid or solid excipients in cold liquefied propellants such as, for example, fluorohydrocarbons, in particular TG227 (2H-heptafluoropropane), TG134a (1,1,1,2-tetrafluoroethane), TG152a (1,1-difluoroethane), TG143a (1,1,1-trifluoroethane) or mixtures thereof or in hydrocarbons such as, for example, butane, isobutane, pentane, hexane, heptane or other readily evaporable liquids such as, for example, ethanol, isopropanol, methanol, propanol.

The suspension obtained is then mixed directly with the carrier material, the carrier material or a number of carrier materials or carrier material/excipient mixtures being introduced dry or in suspension or the active compounds being introduced in suspension with or without excipients. The carrier material suspensions or mixtures can also be added to the active compound suspension or the suspensions of active compound/excipient mixtures. By means of subsequent evaporation of the suspending medium in suitable evaporation vessels or evaporation apparatuses, for example having an inserted or permanently installed stirring device and/or built-in product stripping-off arrangements, the powder(s) are thus applied to the carrier materials or corresponding mixtures in order to obtain the dry powder formulations. Furthermore, it is also possible to isolate an active compound or described mixtures micronized in a bead mill firstly by evaporation of the suspending medium as bulk material in order then to use it, if required, for the preparation of dry powder mixtures after prior resuspension and reagglomeration of the particles, for example, by means of Ultraturrax (IKA), colloid mill, mixer reactor (Broglie or Becomix). Micronized powders isolated beforehand can likewise be applied to carrier materials or mixtures as in the process described above after suspension in suitable suspending media. This may be necessary if, for example, the active compound or mixtures and the carrier materials or mixtures are soluble in the suspending medium. It can then be micronized in the one suspending medium and, after the substance isolation, the powder obtained can be applied in the manner described, such as, for example, shown in example 2 or 3 or 4 or 5, to the carrier material or its mixtures or active compound/carrier material mixtures or active compound/carrier material/excipient mixtures in another suspending medium.

Further active substances which can be employed in the processes mentioned (micronization and/or application process) are, for example: analgesics, antiallergics, antibiotics, anticholinergics, antihistamines, substances having antiinflammatory activity, antitussives, bronchodilators, diuretics, enzymes, substances having cardiovascular activity, hormones. Examples of analgesics are: codeine, diamorphine, dihydromorphine, ergotamine, fentanyl, morphine; examples of antiallergics are: cromoglycic acid, nedocromil; examples of antibiotics are cephalosporins, fusafungin, neomycin, penicillins, pentamidine, streptomycin, sulfonamides, tetracyclines; examples of anticholinergics are: atropine, atropine methonitrate, ipratropium, tiotropium, oxitropium, trospium; examples of antihistamines are: azelastine, methapyrilene; examples of substances having antiinflammatory activity are: beclomethasone, budesonide, dexamethasone, flunisolide, fluticasone, tripredane, triamcinolone; examples of antitussives are narcotine, noscapine; examples of bronchodilators are bambuterol, bitolterol, carbuterol, clenbuterol, formoterol, fenoterol, hexoprenaline, ibuterol, isoprenaline, isoproterenol, metaproterenol, orciprenaline, phenylephrine, phenylpropanolamine, pirbuterol, procaterol, reproterol, rimiterol, salbutamol, salmeterol, sulfonterol, terbutaline, tolobuterol; examples of diuretics are amiloride, furosemide; examples of substances having cardiovascular activity are: diltiazem and nitroglycerin; an example of an enzyme is trypsin; examples of hormones are cortisone, hydrocortisone, prednisolone testosterone, cstradiol; examples of proteins and peptides are abarelix, buserelin, cetrorelix, leuprolide, cyclosporine, ganirelix, glucagon, lutropin (LH), insulin, ramorelix, teverelix (Antarelix®). The examples mentioned can be employed as free acids or bases or as salts. Counterions which can be employed are, for example, physiologically tolerable alkaline earth or alkali metals or amines and also, for example, acetate, adipate, ascorbate, alginate, benzoate, benzenesulfonate, bromide, carbonate, carboxymethylcellulose (free acid), citrate, chloride, dibutylphosphate, dihydrogencitrate, dioctylphosphate, dihexadecylphosphate, fumarate, gluconate, glucuronate, glutamate, hydrogencarbonate, hydrogentartrate, hydrochloride, hydrogencitrate, iodide, lactate, alpha-lipoic acid, malate, maleate, pamoate, palmitate, phosphate, salicylate, stearate, succinate, sulfate, tartrate, tannate, oleate, octylphosphate. Esters can also be employed, for example acetate, acetonide, propionate, dipropionate, valerate. Carrier materials which can be employed are, for example, lactose, dextrose, sorbitol, polyalcohols, sorbitol [sic] mannitol, xylitol, disaccharides such as, for example, maltose and trehalose and polysaccharides such as, for example, starch and its derivatives, oligosaccharides such as, for example, cyclodextrins, and also dextrins and various amino acids. Excipients which can be employed are the carrier materials just mentioned and also, preferably, the amino acid leucine individually or in the form of a mixture, in each case in micronized or coarse form or as a lyophilizate (lyophilizate of excipient solutions or active compound/excipient solutions) with subsequent micronization in suspension (with or without subsequent isolation of the powders) and, for example, lipids such as glyceryl monostearate, glyceryl tristearate, glyceryl tripalmitate and, for example, phospholipids such as, for example, egg lecithin, soybean lecithin, and also vitamins such as, for example, tocopherol acetate (vitamin E) and also surfactants such as, for example, polyoxyethylene sorbitan oletate [sic] or polyoxyethylene sorbitan stearate, preferably solid surfactants such as, for example, Pluronic (R) F68 (Fluka) or solid polymers such as, for example, polyethylene glycol 2000 or polyethylene glycol 4000.

The excipients mentioned can be soluble, partially soluble or insoluble in the suspending medium. In the case of solubility or partial solubility, a coating of the ground particle or a coating of the carrier particles loaded with active compound could be carried out.

The powders or powder formulations which can be prepared by the application process are suitable, for example, for direct use in powder inhalers such as, for example, MDPIs, blister inhalers.

The powders or powder mixtures which can be prepared by the micronization process are suitable, for example, directly as suspensions or alternatively after isolation of the powders and subsequent resuspension in metered-dose aerosols or for the preparation of dry powders for other pharmaceutical purposes, such as, for example, tableting and also for further applications in which micronized powders are needed.

A micronized powder or a micronized powder mixture prepared using the bead mill shows the following advantages compared with a mixture prepared dry:

Powders are less contaminated compared, for example, with a spray-dried product (for example the small increase in peptide contamination, see example 1)

Powders are very fine, 90% of the particles are, for example, smaller than 4.9 μm (FIG. 2 from example 1) and thus suitable for the preparation of inhalable powder formulations having local and systemic therapeutic activity.

The micronization of active compounds or excipients or mixtures thereof in liquids shows the following advantages, compared with other micronization processes:

The grinding chamber can be cooled to −60° C., which makes possible grinding in liquid propellant (for example TG227 and TG134a) or other liquids (e.g. ethanol, butane, and other readily evaporable liquids mentioned in this patent specification) and their possible mixtures at normal pressure. Under this condition, soft substances can be more easily ground, since they are more brittle at low temperatures.

By grinding at low temperatures, for example <−30° C., preferably <−40° C., but more preferably at <−50° C., these powders are chemically less contaminated. Denaturation problems in the presence of water, for example in the case of peptides peptide hydrolysis, deamidation, Mallard reaction with reducible sugars etc., or oxidation of oxidation-sensitive substances are avoided or occur to a more insignificant extent than at room temperature.

Active compounds or mixtures or active compound/excipient mixtures can be ground, for example, rapidly and effectively in high yield to the desired particle sizes, preferably <5 μm, but more preferably <3 μm, as highly concentrated suspensions. According to the particular requirement, the concentrated suspension can be diluted, mixed with other suspensions or with dry powders, and then evaporated.

By the use of, for example, iridium or yttrium stabilized ZrO₂ grinding beads and ZrO₂-coated static and rotating parts of the grinding system, a high resistance to abrasion is achieved and a powder (or suspension) of pharmaceutical quality (purity) is obtained (Federal Ministry for Employment and Social Affairs, technical rules for hazardous substances 900 (TRGS 900): threshold values in air at the workplace “atmospheric threshold values”. Federal employment bulletin (BarbBl.) Issue 10.1996; and supplements: BarbBl. 11/1997, p. 39; BarbBl. 5/1998, p. 63; BarbBl. 10.1998, p. 73).

The suspensions obtained by micronization can be prepared by various evaporation methods either as pure micronized active compound powders or as surface-modified powders.

Lower danger of an electrostatic charge on the product during grinding, since this is present in suspended form and thus no dust occurs. This thus also means a lower danger of environmental contamination due to the closed system.

The application process (application of micronized powders in suspension to carrier materials or mixtures) shows the following advantages compared with the dry mix process:

Simple preparation of active compound- or excipient-loaded carrier materials.

The active compound is applied more uniformly to the carrier material or carrier material mixtures, thus better dosage accuracy of the finished powder formulation.

Combination preparations can be prepared more easily and in a shorter time than in the case of the dry mix method, since they can be dispersed together in the suspending medium or ground together in a bead mill and can be applied to the carrier materials in suspension. They do not have to be prepared by means of many individual sieving and mixing steps.

By the use of, for example, propellants such as TG227, an anhydrous preparation of hygroscopic powder formulations such as, for example, in the case of formulations containing cromoglycic acid, is made possible.

Practical applications in the pharmaceutical field are, for example:

Micronization of active compounds and/or excipients in liquid propellant and subsequent evaporation of the propellant. As a result, micronized pure dry powders can be prepared, e.g. for MDPI use or for the preparation of injectable very fine suspensions and also for all pharmaceutical applications where a micronized powder would be advantageous, such as, for example, for inhalatory purposes by means of a blister inhaler or in tableting.

Preparation of particles having modified surface properties by dissolving or suspending an excipient directly in the suspension either before or after micronization and evaporating the solvent.

Preparation of particles having modified surface properties by dissolving one or more excipients in a suitable solvent with the active compound and then obtaining by, for example, lyophilization a homogenous mixture of, for example, lactose and/or leucine with, for example, formoterol. This can be micronized using a bead mill to particle sizes of below 0.1 μm to 5 μm, preferably to 0.2 to 4 μm, but more preferably to 0.3 to 3 μm and this suspension can be applied to coarse carrier materials such as, for example, pourable lactoses (for example having particle sizes of 10-900 μm) by the application process mentioned.

The powders or powder mixtures obtained according to the invention can be conditioned by processes known per se (e.g. allowing to stand at 25° C. and 60% rel. humidity for a few hours to several days) to avoid electrostatic charge.

In the indication of particle sizes, the d(10%) value here is always intended for the lower particle size range and the d(90%) value for the tipper particle size range. For example, a particle size of 0.3-3 μm here means: 10% of the particles are smaller than 0.3 μm and 90% of the particles are smaller than 3 μm.

FIG. 1 shows a sketch of the modified bead mill in cross-section

FIG. 2 shows the particle size distribution of the cetrorelix acetate micronized in example 1, measured by means of laser diffractometry (Malvern Mastersizer)

FIG. 3 shows a scanning electron micrograph of a section of the particles prepared in example 2 which have been applied to SperoLac 100 in suspension.

FIG. 4 shows a scanning electron micrograph of a section of the particles prepared as a comparison in comparison example 2a, which have been applied dry to SperoLac [sic] 100, i.e. according to the conventional process.

The invention—the preparation of powder formulations by micronization of the active compound and subsequent loading of the carrier material with the micronized active compound—is illustrated in greater detail with the aid of the following working examples without being restricted thereto:

### EXAMPLE 1

### Obtainment of the Powder

In a modified SL-12C bead mill from VMA-Getzmann, wet grinding of cetrorelix acetate in liquid HFA 227 was carried out in combination with a cryostat (from Haake, mod. No.: N8-KT9OW with a PT35/170-140 centrifugal pump). For this, 100 ml of iridium-stabilized zirconium dioxide grinding beads (having 0.6 mm diameter) were introduced into the grinding chamber. The isolated double jacket of the grinding chamber and the isolated reservoir of the bead mill were connected to the cryostat and cooled to −60° C. The bead mill was rinsed twice with 150 ml each of ethanol (100%) at a speed of rotation of the rotor of 6 m/s. The apparatus was then rinsed with 200 ml of HFA 227. The rinsing liquids were discarded.

500 g of HFA 227 were introduced into the bead mill and the system was adjusted to a temperature of −50° C. (the reflux temperature of the suspension −35° C.). 40 g of cetrorelix acetate were then predispersed in 500 g of BFA 227 with the aid of an Ultraturrax (at 8000 min⁻¹; for 2 min). This suspension was added to the bead mill at a speed of rotation of the rotor of 5.5 m/s in the course of 1 min. The suspension was ground at 5.5 m/s for 5 min, at 7 m/s for 15 min and then ground at 13.5 m/s for 10 min. At the end, the temperature remained unchanged. After grinding had been completed, the suspension was filled into a 1 liter round-bottomed flask and the propellant was evaporated in the course of 1 h with rotation of the flask at 200 min⁻¹ with gentle boiling. The white powder obtained was then dispensed into a 100 ml glass screw bottle. The particle diameter was determined by means of laser diffractometry. 90% (d 0.9) of the particles were <4.9 μm (see FIG. 2). The volume mean diameter (VMD) was 2.5 μm. The peptide impurities determined by means of HPLC were increased only by 0.08% by the grinding process. The inorganic impurities with respect to zirconium dioxide (ceramic abrasion) were 96 μg/g in the solid.

### EXAMPLE 2

### Mixing in Suspension (SpheroLac 100)

200 g of liquid TG227 (temp. −50° C.) were introduced into a 250 ml beaker. 1.03(4) g of the cetrorelix acetate obtained from example 1 were then slowly added to this and the mixture was dispersed at 22 000 min⁻¹ for 1 min using an Ultraturrax. After removal of the Ultraturrax, the cetrorelix acetate suspension was added to a suspension consisting of 8.96(6) g of SpheroLac 100 (Meggle Pharma) and 50 g of HFA 227. This total mixture was evaporated within 1 h with rotation of the flask at 200 min⁻¹ with gentle boiling of the suspension. The pourable cetrorelix acetate/lactose mixture obtained was then dispensed into a 30 ml glass screw bottle. 1 g each of the powder was then dispensed into MDPI cartridges (cartridges for the Novolizer®). The determination of the inhalable fraction of the powder mixture obtained was determined [sic] in a cascade impacter (multi-stage liquid impinger, Astra) at a flow rate of 70 liters of air/min using the Novolizer® (MDPI) as the disperser unit. For this, a cartridge filled with the powder mixture was employed in the Novolizer®. The inhaler was mounted on the cascade impacter and triggered. The content determinations in the individual stages of, the cascade impacter determined by HPLC were used for the determination of the respirable fraction (cascade 3-5). The fraction here was 41% (n=1)

### COMPARISION EXAMPLE 2a

### Dry Mixture (SpheroLac 100)

1.03(4) g of the cetrorelix acetate obtained from example 1 were premixed for 5 min with 8.96(6) g of SpheroLac 100 (Meggle Pharma) in a glass screw bottle in the Turbula mixer. The mixture was then compulsorily sieved through a 315 μm stainless-steel analysis sieve (10 cm diameter) with the aid of 1 g of iridium-stabilized zirconium oxide grinding beads of 1.1 mm diameter. The mixture obtained was dispensed into a glass screw bottle and mixed in the Turbula mixer for 30 min. 1 g each of the powder was then dispensed into MDPI cartridges (cartridges for the Novolizer®). The determination of the inhalable fraction of the powder mixture obtained was carried out as described above (see tab. 1).

### EXAMPLE 3

### Mixture in Suspension (CapsuLac 60 Plus Turbula)

200 g of liquid TG227 (temp. −50° C.) were introduced into a 250 ml beaker. 1.97(2) g of the cetrorelix acetate obtained from example 1 were then slowly added to this and the mixture was dispersed for 1 min at 22000 min⁻¹ using an ultraturrax. After removal of the ultraturrax, the suspension was added to a round-bottomed flask containing 18.03 g of CapsuLac 60. This mixture was evaporated in the course of 1 h with rotation of the flask at 200 min⁻¹ with gentle boiling of the suspension. The pourable cetrorelix acetate/lactose mixture obtained was then dispensed into a 30 ml glass screw bottle and mixed for 30 min in the Turbula mixer. 1 g each of the powder mixture was then dispensed into MDPI cartridges (cartridges for the Novolizer®). The determination of the inhalable fraction of the powder mixture obtained was carried out as described in example 2 (see tab. 1).

### COMPARISON EXAMPLE 3a

### Dry Mixture (CapsuLac 60)

For this, 1.972 g of the cetrorelix acetate obtained from example 1 were premixed for 5 min with 18.03(2) g of CapsuLac 60 (Meggle Pharma) in a glass screw bottle in the Turbula mixer. The mixture was then compulsorily sieved though a 315 μm stainless-steel analysis sieve (10 cm diameter) with the aid of 1 g of iridium-stabilized zirconium oxide grinding beads of 1.1 mm diameter. The mixture obtained was dispensed into a glass screw bottle and mixed in the Turbula mixer for 30 min. 1 g each of the powder was then dispensed into MDPI cartridges (cartridges for the Novalizer®). The determination of the inhalable fraction of the powder mixture obtained was carried out as described in example 2 (see tab. 1).

### EXAMPLE 4

### Mixture in Suspension (CapsuLac 60)

200 g of liquid TG227 (temp. −50° C.) were introduced into a 250 ml beaker. 1.97(2) g of the cetrorelix acetate obtained from example 1 were then slowly added to this and the mixture was dispersed for 1 mm at 22 000 min⁻¹ using an ultraturrax. After removal of the ultraturrax, the cetrorelix acetate suspension was added in a round-bottomed flask to a suspension consisting of 18.03 g of CapsuLac 60 (Meggle Pharma) and 50 g of HFA 227. This total mixture was evaporated in the course of 1 h with rotation of the flask at 200 min⁻¹ with gentle boiling of the suspension. The pourable cetrorelix acetate/lactose mixture obtained was then dispensed into a 30 ml glass screw bottle. 1 g each of the powder mixture was then dispensed into MDPI cartridges (cartridges for the Novalizer®). The determination of the inhalable fraction of the powder mixture obtained was carried out as described in example 2 (see tab. 1). As a comparison thereto, the dry mixture of cetrorelix acetate with CapsuLac 60 from example 3a was used.

### EXAMPLE 5

### Mixture in Suspension (CapsuLac 60)

210 g of liquid TG227 (temp. −50° C.) were weighed into a 250 ml beaker. The propellant was then slowly added to 422 mg of micronized budesonide and the mixture was dispersed at 22000 min⁻¹ for 30 sec using an ultraturrax. After removal of the ultraturrax, the budesonide suspension was added in a round-bottomed flask to a suspension consisting of 22.58 g of CapsuLac 60 (Meggle Pharma) and 50 g of HFA 227. This total mixture was evaporated in the course of 1 h with rotation of the flask at 60 min⁻¹ with gentle boiling of the suspension. Powder adhering to the glass wall was dissolved by tapping. The solution was then dried for 10 min by applying a vacuum (20 to 30 mbar). The flask rotated at 60 min⁻¹ for a further 30 min. The pourable budesonide/lactose mixture obtained was then dispensed into a 50 ml glass screw bottle. 1-1.5 g each of the powder mixture was then dispensed into MDPI cartridges (cartridges for the Novolizer®). The determination of the inhalable fraction of the powder mixture obtained was carried out as described in example 2 (see tab. 1).

### COMPARISON EXAMPLE 5a

### Dry Mixture (CapsuLac 60)

4.22 g of budesonide were mixed in the Turbula (centrifugal mixer) for 10 min with 135.5 g of CapsuLac. This premixture was sieved through a stainless-steel sieve and added to 90.3 g of CapsuLac. This mixture in turn was dispensed into a glass screw bottle and mixed in the turbula mixer for 30 min. 1-1.5 g each of the powder was then dispensed into MDPI cartridges (cartridges for the Novolizer®). The determination of the inhalable fraction of the powder mixture obtained was carried out as described in example 2 (see tab. 1).

1. Process for the preparation of fine-particulate sensitive substances or substance mixtures, in particular of proteins, which essentially have particle sizes in the nano(nm)- to micrometer(μm) range, characterized by the steps
a) grinding of the substance or of the substance mixture in a suspending medium in which the substance or the substance mixture is largely insoluble, at low temperature and
b) subsequent removal of the suspending medium.

2. Process according to claim 1, characterized in that the suspending medium is selected from the group consisting of unsubstituted hydrocarbons, hydrocarbons which are mono- or polysubstituted by fluorine atoms and mixtures thereof.

3. Process according to claim 1 or 2, characterized in that the suspending medium is a hydrocarbon which is mono- or polysubstituted by fluorine atoms, selected from the group consisting of TG227, TG134a, TG152a, TG143a and mixtures thereof.

4. Process according to one of the above claims, characterized in that the suspending medium is an unsubstituted hydrocarbon selected from the group consisting of butane, isobutane, pentane, hexane, heptane or mixtures thereof.

5. Process according to one of the above claims, characterized in that the suspending medium is selected from the group consisting of butane, isobutane, pentane, hexane, heptane, TG227, TG134a, TG134a, TG152a, TG143a or mixtures thereof.

6. Process according to one of the above claims, characterized in that the grinding is carried out at approximately a temperature T selected from the group consisting of T £−30° C., T £−40° C., T £−50° C. and T £−60° C.

7. Process according to one of the above claims, characterized in that before and/or after the grinding of the suspension an excipient in each case independently of one another selected from the group consisting of lactose, dextrose, sorbitol, mannitol, polyalcohol, xylitol, disaccharides, polysaccharides, oligosaccharides, dextrins, amino acids, solid lipids, solid phospholipids, vitamins, surfactants, polymers and mixtures thereof is added.

8. Process according to one of the above claims, characterized in that the substance to be ground has been selected from the group consisting of the peptides abarelix, buserelin, cetrorelix, leuprolide, cyclosporine, ganirelix, glucagon, lutropin (LH), insulin, ramorelix, teverelix (Antarelix).

9. Solid fine-particulate pharmaceutical preparation comprising an active compound or an active compound combination, in particular for inhalatory administration in mammals, obtainable by the process according to claims 1 to 7.

10. Solid preparation according to claim 9, characterized in that at least one active compound is selected from the group consisting of the peptides abarelix, buserelin, cetrorelix, leuprolide, cyclosporine, ganirelix, glucagon, lutropin (LH), insulin, ramorelix, teverelix (Antarelix).

11. Solid preparation according to claim 9 or 10 for use in powder inhalers such as, for example, DPI, MDPI or blister inhalers.

12. Process for the application of fine-particulate substances or substance mixtures to carrier materials, characterized in that the suspending medium is stripped off a suspension comprising the fine-particulate substance or the fine-particulate substance mixture, the carrier materials and the suspending medium in which both the substance or the substance mixture and the carrier materials are largely insoluble with thorough mixing.

13. Process according to claim 12, characterized in that the suspending medium consists of substances or substance mixtures which are gaseous at room temperature and under normal pressure.

14. Process according to claim 12 or 13, characterized in that the suspending medium is selected from the group consisting of unsubstituted hydrocarbons or hydrocarbons which are mono- or polysubstituted by fluorine atoms or mixtures thereof.

15. Process according to one of the above claims 12-14, characterized in that the suspending medium is selected from the group consisting of butane, isobutane, pentane, hexane, heptane, TG227, TG134a, TG152a, TG143a or mixtures thereof.

16. Process according to one of the above claims 12-15, characterized in that the suspending medium is selected from the group consisting of TG227, TG134a, TG152a, TG143a or mixtures thereof.

17. Process according to one of the above claims 12-16, characterized in that the carrier material is selected from the group consisting of spherical and/or agglomerated lactose, the spherical lactose having a smooth surface and the agglomerated lactose having a rough surface.

18. Process according to one of the above claims 12-17, characterized in that the fine-particulate substance or the fine-particulate substance mixture has an average particle size of approximately 0.1-10 μm and the carrier material has an average particle size of approximately 10-900 μm.

19. Process according to one of the above claims 12-18, characterized in that one or more excipients selected from the group consisting of lactose, dextrose, sorbitol, mannitol, polyalcohol, xylitol, disaccharides, polysaccharides, oligosaccharides, dextrins, amino acids, solid lipids, solid phospholipids, vitamins, surfactants, polymers and mixtures thereof are additionally present in the suspension.

20. Process according to one of the above claims 12-19, characterized in that the carrier material is added to the suspension obtained by process step a) according to one of claims 1 to 8 and the suspending medium is then removed.

21. Solid pharmaceutical preparation comprising an active compound or an active compound combination, in particular for inhalative administration in mammals, obtainable by one of the processes according to claims 12 to 19.

22. Solid preparation according to claim 21, characterized in that at least one active compound is selected from the group consisting of the peptides abarelix, buserelin, cetrorelix, leuprolide, cyclosporine, ganirelix, glucagon, lutropin (LH), insulin, ramorelix, teverelix (Antarelix).

23. Solid preparation according to claim 21 or 22 for use in powder inhalers such as, for example, DPI, MDPI or blister inhalers.
